(19) United States
(12) Patent Application Publication
(10) Pub. No.: US 2011/0236946 A1
(43) Pub. Date: Sep. 29, 2011

(54) Concurrent Anaerobic Digestion and Fermentation of Lignocellulosic Feedstocks

(75) Inventors: John Ross Maclachlan, Burnaby (CA); Edward Kendall Pye, Burnaby, PA (US)

(73) Assignee: LIGNOL INNOVATIONS LTD., Burnaby, BC (CA)

(21) Appl. No.: 12/602,036

(22) PCT Filed: May 23, 2008
(86) PCT No.: PCT/CA08/01001

§ 371 Date: Jul. 26, 2010

Related U.S. Application Data
(60) Provisional application No. 60/941,197, filed on May 31, 2007.

(51) Int. Cl. C12P 5/02; C12M 1/00

(52) U.S. Cl. 435/167; 435/289.1

(57) ABSTRACT

A process for concurrent production of lignins, fuel alcohol, and biogas from lignocellulosic feedstocks. The process comprises: (1) pretreating a lignocellulosic feedstock to produce a solubilised liquid components stream comprising lignins, lignin-derived compounds, and a cellulosic pulp stream, (2) separating the liquid stream from the cellulosic pulp stream, (3) processing the liquid stream to separate and recover at least lignins, lignin-derived compounds, and semi-solid waste material, (b) processing the cellulosic pulp stream to saccharify and ferment the cellulose pulp to produce a beer which is then separated into fuel-grade alcohol and a waste stillage material, (4) anaerobically digesting the semi-solid waste material from the liquid stream and the waste stillage material to produce a biogas. The rate of anaerobic digestion can be manipulated by controllably supplying a portion of the monosaccharides produced from the cellulosic pulp. The cellulosic pulp stream may also be anaerobically digested.

### TECHNICAL FIELD

This invention relates to systems and methods for production of combustible fuels from fibrous biomass. More particularly, this invention relates to manipulable concurrent production of biogas, fuel alcohol, organic acids and chemicals from lignocellulosic feedstocks.

### BACKGROUND ART

The industrial and commercial benefits of anaerobic digestion systems include, in addition to the production of biogas useful for cogeneration of heat and electrical power, the provision of energy and cost-efficient in-house wastewater treatment of industrial effluents. However, the disadvantages include lengthy digestion times due to the biological nature of the process stages, and further delays or inhibition of the biological processes caused by adverse effects of certain constituents of organic waste streams on microbial enzyme systems. Digestion rates in anaerobic systems configured for processing organic wastes and materials, are often significantly reduced due to the lack of enzymes necessary for complete digestion. This lack of enzymes can be attributed to: (1) poor growth of the bacteria which produce these enzymes; (2) the lack of access of the appropriate and acclimated bacteria to the feedstock; (3) feedback inhibition of enzyme production due to accumulating byproducts in intimate contact with the bacterial cells; and (4) inhibition of enzyme activity can be due to high concentrations of byproduct intermediates in the fermentation fluid. Low rates of digestion can also be due to fresh feedstock slurries displacing settled slurries containing aggregated populations of the active enzyme-producing bacteria. Anaerobic digestion systems are commonly employed for municipal and industrial conversion of organic wastes into biogases that are subsequently captured for use in heat and/or electrical power generation. Anaerobic conversion of organic wastes into biogases generally occurs along a four-stage process comprising (a) a first stage during which complex organic molecules are hydrolyzed into soluble monomers such as monosaccharides, amino acids and fatty acids (i.e., hydrolysis), followed by (b) a second stage during which the simple monomers produced during the first stage, are converted into volatile fatty acids (i.e., acidogenesis), then (c) a third stage during which the volatile fatty acids are converted into acetic acid, CO₂, and hydrogen (i.e., acetogenesis), and finally (d) the fourth stage where the acetic acid is converted into methane, CO₂, and water (methanogenesis). Biogas produced by such anaerobic conversions comprises primarily methane and secondarily CO₂, and trace amounts of nitrogen gas, hydrogen, oxygen and hydrogen sulfide.

The four stages of anaerobic digestion are microbially mediated and each stage of anaerobic digestion typically involves different types of naturally occurring synergistic anaerobic bacteria. Large-scale anaerobic digestion systems may be configured to separate the four stages into separate vessels, e.g., in continuous throughput systems, and supplement each vessel with inocula of selected suitable microbial cultures to optimize the conversion efficiency of each stage. Alternatively, it is also possible to maintain all for stages of anaerobic digestion within one vessel, e.g., in batch systems, by providing inocula comprising the four groups of anaerobic bacteria. Exemplary hydrolytic bacteria are Enterobacter sp., exemplary acidogenic bacteria include Bacillus sp., Lactobacillus sp. and Streptococcus sp., exemplary acetogenic bateria include Acetobacter sp., Gluconobacter sp., and certain Clostridium sp., while exemplary methogenic bacteria are from the Methanobacteria, Methanococci, and Methanopyri genera.

The most common major polymeric component of organic wastes is cellulose, and it is known that microbial hydrolysis of cellulose is the most significant rate-limiting step during the first stage of anaerobic digestion subsequently affecting the throughput speed and efficiencies of the remaining stages (Adney et al., 1991, Appl. Biochem. Biotechnol. 30:165-183; Yingnan et al., 2004, Bioresour. Technol. 94: 197-201). Cellulosic materials commonly present in organic waste streams typically contain significant amounts of lignin. Lignin-derived polymeric materials are particularly recalcitrant in anaerobic digestion systems and are often directly responsible for anaerobic enzyme system inhibition. It is known that lignin-derived waste streams (termed “black liquors” or “spent liquors” by those skilled in these arts) from pulping processes are not amenable for anaerobic digestion because of the inhibitory effects of lignins on anaerobic metabolism (Peng et al., J. Chem. Tech. Biotechnol. 1993, 58: 89-93). Furthermore, it appears that methanogenic bacteria in particular, are adversely affected by lignins (Yin et al., 2000, Biotechnol. Lett. 22: 1531-1535).

### DISCLOSURE OF THE INVENTION

Exemplary embodiments of the present invention are directed to processes and systems configured for separating lignocellulosic feedstocks into (a) a liquid stream comprising solubilised components, and lignins and lignin-derived polymers, and (b) an amorphous de-lignified solids output stream comprising cellulosic pulp. The liquid components stream contains at least lignins, lignin-derived polymers, hemicelluloses, oligosaccharides, polysaccharides, monosaccharides and spent solvent. The liquid components stream is processed to recover at least two separate classes of lignins, to recover and recharge the spent solvent for recycling, to additionally separate at least furfural, sugar syrups, organic acids and a semi-solid waste material. The cellulosic pulps are useful for production of fuel alcohol, biogas, fermentation products, fine chemicals, cellulose powders, cellulose derivatives, and high-quality paper products. At least the semi-solid waste material produced during processing of the liquid components stream is anaerobically digested to produce biogas. The anaerobic digestion is a four-step/component process wherein the first step is liquefaction of the semi-solid waste material, the second step is acidification of the liquefied waste material, the third step is acetification of the acidified liquefied waste material, and the fourth step is conversion of the acetic acid to biogas (i.e., methane and carbon dioxide) plus water and a mineral residue.

One exemplary embodiment of the present invention is directed to the concurrent production of fuel alcohol and biogas from lignocellulosic feedstocks. The lignocellulosic feedstocks are separated into an amorphous mostly de-lignified solids output stream comprising cellulosic pulp, and a liquid stream comprising solubilised components. The cellulosic pulp is hydrolyzed into a monosaccharide sugar stream which is then fermented into a beer. The beer is distilled to produce a fuel-grade alcohol and a stillage.

According to one aspect, the stillage is anaerobically digested to produce biogas.

According to another aspect, a portion of the monosaccharide sugar stream produced during hydrolysis of the cellulosic pulp is controllably provided to the anaerobic process to affect the rate of biogas production.

According to a further aspect, selected portions of the liquefied waste material are controllably provided to the processing steps for the liquid components stream to increase the amounts of sugars, furfurals and organic acids recovered from the lignocellulosic feedstocks.

Another exemplary embodiment of the present invention is directed to a lignin biorefinery for lignocellulosic feedstocks wherein the output products are separated classes of lignins, other organic components extracted from the lignocellulosic feedstocks, and biogas. After pre-treatment of the lignocellulosic feedstocks to produce an amorphous de-lignified solids output stream comprising cellulosic pulp, and a liquid stream comprising solubilised components, the cellulosic pulp is anaerobically digested. The liquid components stream is processed to recover at least two separate classes of lignins, to recover and recharge the spent solvent for recycling, to additionally separate at least furfurals, sugar syrups, organic acids and a semi-solid waste material.

### BRIEF DESCRIPTION OF THE DRAWINGS

The present invention will be described in conjunction with reference to the following drawings in which:

FIG. 1 is a schematic flowchart of an exemplary embodiment of the present invention illustrating a modular continuous counter-flow system for processing a lignocellulosic feedstock with interactive and cooperating fermentation and anaerobic digestion modules;

FIG. 2 is a schematic flowchart of the system from FIG. 1 illustrating an exemplary configuration of a suitable 4-stage anaerobic digestion module; and

FIG. 3 is schematic flowchart showing another exemplary embodiment of the present invention illustrating a modular lignin biorefinery system configured for processing a lignocellulosic feedstock into: (a) a liquid extractives stream from which three classes of lignin compounds may be separated and recovered, and (b) a solids stream which is processed by anaerobic digestion to produce a fourth class of lignin compounds, biogas, mineralized solids and water, and optionally, monosaccharides and organic acids which may be routed back to the liquid extractives stream for purification and recovery.

### DETAILED DESCRIPTION OF THE INVENTION

Exemplary embodiments of the present invention are directed to processes, systems and equipment configured for separating lignocellulosic feedstocks into multiple output streams. At least one stream produced is a liquid stream comprising solubilised extractives comprising at least lignins and lignin-derived polymers, hemicelluloses, polysaccharides, oligosaccharides furfurals and phenolic compounds, At least one other stream produced is a solids stream comprising cellulosic pulps. Suitable lignocellulosic feedstocks are exemplified by angiosperm fibrous biomass, gymnosperm fibrous biomass, field crop fibrous biomass, waste paper and wood materials, the like, and mixtures thereof.

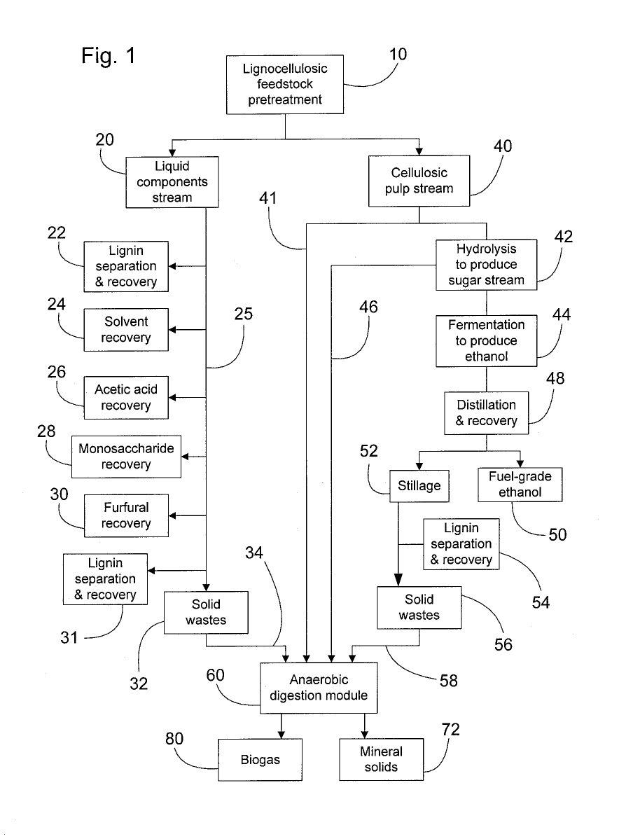

Suitable processes and processing systems for separating lignocellulosic feedstocks into liquid streams comprising lignins, saccharides, oligosaccharides and polysaccharides, and solids streams comprising cellulosic pulps, are exemplified by biorefining, thermochemical and/or chemical and/or enzymatic pulping processes and systems. A suitable exemplary pulping system is shown in FIG. 1 and is based on pretreating lignocellulosic feedstocks 10 by perfusing and cooking at suitably elevated temperatures, physically disrupted and comminuted fibrous feedstocks in aqueous organic solvents thereby producing solid amorphous pulp materials and spent solvents. Suitable aqueous organic solvents are exemplified by ethanol diluted in water with an inorganic or alternatively, an organic acid provided as a reaction catalyst. An exemplary inorganic acid is sulfuric acid. The amorphous pulp materials thus produced primarily comprise purified cellulose-rich fibers that are low in residual lignin and in which the cellulose crystallinity has been significantly reduced. The spent solvents are commonly referred to as black liquors, and typically comprise solubilized lignins and lignin-derived polymers, furfural, xylose, acetic acid, lipophylic extractives, other monosaccharides, oligosaccharides and spent ethanol. The solid amorphous cellulosic pulp material is separated into a cellulosic pulp stream 40 and black liquor liquid components stream 20.

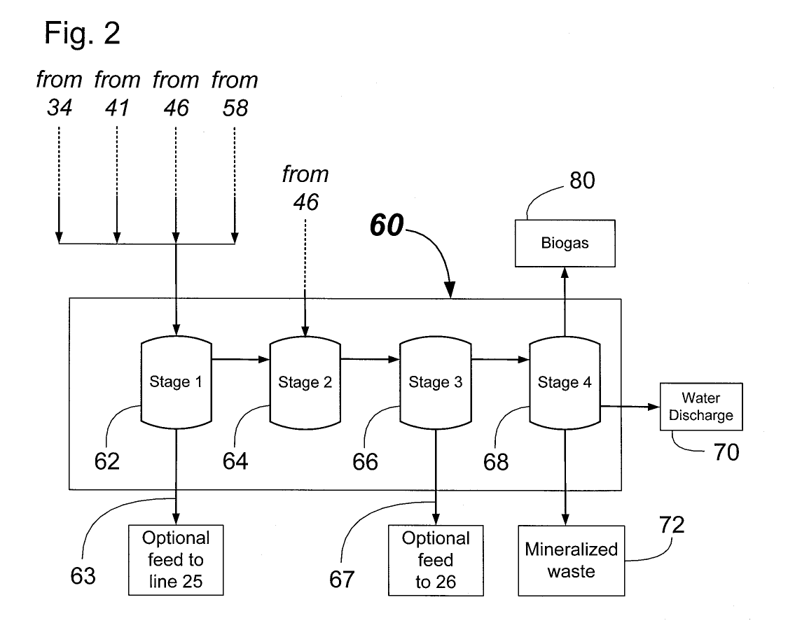

The liquid components stream 20 is processed to sequentially separate and remove at least two distinct classes of lignins and lignin-derived polymers 22 (i.e., medium-molecular weight lignins and low-molecular weight lignins) by first flashing the stream to atmospheric pressure and then rapidly diluting the black liquor with water thereby causing the lignins and lignin-derived polymers to precipitate out of solutions. The lignins are then removed for further purification and/or processing. The spent solvent is then recovered 24 from the delignified liquid stream, for example by distillation, to make it useful for recycling to the lignocellulosic feedstock pretreatment step 10. The stillage 25 remaining after solvent recovery and distillation 24 may then be further processed to separate therefrom other solubilized components extracted from the lignocellulosic feedstock, such as furfural 30, monosaccharides exemplified by xylose 28, organic acids exemplified by acetic acid 26, and a novel third class of lignins and lignin-derived polymers 31 (i.e., very-low molecular weight lignins). All that is left after these series of steps is a first semi-solid waste material 32. The semi-solid waste material 32 resulting from the processing of the liquids component stream 20 is transferred via transfer line 34 into the Stage 1 vessel 62 of the anaerobic digestion module 60 (FIGS. 1 and 2).

The cellulosic pulp stream 40 may be converted to ethanol or any other fermentation product such as butanol or propanol, by enzymatic hydrolysis to produce a monosaccharide sugar stream 42 which may then be fermented to produce a beer comprising ethanol and fermentative microbial biomass 44. The beer is distilled 48 or otherwise separated to produce a fuel-grade alcohol 80 and a stillage 52. The stillage 52 may be processed to recover therefrom a novel class of lignins and lignin-derived polymers 54 (high-molecular weight lignins), and leaving a second solid waste material 56. The solid waste material 56 resulting from the processing of the cellulosic pulp stream 40, is transferred via transfer line 58 into the Stage 1 vessel 62 of the anaerobic digestion module 60 (FIGS. 1 and 2). However, it is optional if so desired, to directly transfer the cellulosic pulp stream 40 produced by the lignocellulosic feedstock treatment 10, via transfer line 41 into the Stage 1 vessel 62 of the anaerobic digestion module 60 (FIGS. 1 and 2). Alternatively, it is within the scope of this invention to recover the cellulosic pulp material for further processing to produce cellulose powders, microcrystalline cellulose, and cellulose derivatives exemplified by CMC-celluslose and DEAE-cellulose.

An exemplary 4-stage anaerobic digestion module 60 according the present invention configured to cooperate and communicate with lignocellulosic feedstock pre-treatment and processing systems is illustrated in FIG. 2. The first stage comprises a sludge tank 62 configured for receiving semi-solid/solid waste materials from one or more of the waste outputs from: (a) the liquid components stream 20 processing via transfer line 34, (b) the lignocellulosic feedstock pre-treatment 10 i.e., the cellulosic pulp stream 40 via transfer line 41, (c) the stillage wastes 56 from the distillation of cellulosic fermentation beer 48 to produce fuel-grade alcohol or other fermentation product 80. The first stage sludge tank 62 may optionally receive: (d) a portion of the monosaccharide sugar stream 42 produced during enzymatic hydrolysis of the cellulosic pulp, via transfer line 46. The sludge tank 62 is maintained under anaerobic conditions to maintain populations of facultative anaerobic bacteria that produce enzymes capable of hydrolyzing the complex molecules comprising waste materials into soluble monomers such as monosaccharides, amino acids and fatty acids. It is within the scope of the present invention to provide if so desired inocula compositions for intermixing and commingling with the semi-solid/solid wastes in the sludge tank 62 to expedite the hydrolysis processes to produce a liquid stream. Suitable hydrolyzing inocula compositions are provided with at least one Enterobacter sp.

The liquid stream produced in the sludge tank 62 is transferred into a second-stage acidification vessel 64 wherein anaerobic conditions and a population of acidogenic bacteria such as Bacillus sp., Lactobacillus sp. and Streptococcus sp. are maintained. It is optional for a portion of the monosaccharide sugar stream 42 produced during enzymatic hydrolysis of the cellulosic pulp, to be delivered into the acidification vessel 64 via transfer line 46. The monosaccharides, amino acids and fatty acids contained in the liquid stream received into the acidification vessel 64 are converted into volatile acids by the acidogenic bacteria. It is within the scope of the present invention to provide if so desired acidification inocula compositions configured for facilitating and expediting the production of solubilized volatile fatty acids in the acidification tank 320. Suitable acidification inocula comprise at least one of a Bacillus sp., Lactobacillus sp. and Streptococcus sp., and optionally, may comprise mixtures of two or more of said bacterial species.

A liquid stream comprising the solubilized volatile fatty acids is transferred from the acidification vessel 64 into a third-stage acetogenesis vessel 66 wherein anaerobic conditions and a population of acetogenic bacteria such as Acetobacter sp., Gluconobacter sp., and Clostridium sp., are maintained. The volatile fatty acids are converted by the acetogenic bacteria into acetic acid, carbon dioxide, and hydrogen. It is within the scope of the present invention to provide if so desired inocula compositions configured for facilitating and expediting the production of acetic acid from the volatile fatty acids delivered in the liquid stream into the acetogenesis vessel 64. Suitable acetification inocula compositions are provided with at least one of Acetobacter sp., Gluconobacter sp., and Clostridium sp., and optionally, may comprise mixtures of two or more of said bacterial species.

The acetic acid, carbon dioxide, and hydrogen are then transferred from the acetogenesis vessel 66 into the biogas vessel 68 wherein the acetic acid is converted into methane, carbon dioxide and water by methanogenic bacteria such as Methanobacteria sp., Methanococci sp., and Methanopyri sp. The composition of the biogas produced in the biogas vessel 68 will vary somewhat with the chemical composition of the lignocellulosic feedstock delivered to module A, but will typically comprise primarily methane and secondarily CO₂, and trace amounts of nitrogen gas, hydrogen, oxygen and hydrogen sulfide. It is within the scope of the present invention to provide if so desired methanogenic inocula compositions configured for facilitating and expediting the conversion of acetic acid to biogas. Suitable methanogenic inocula compositions are provided with at least one of bacteria from the Methanobacteria sp., Methanococci sp., and Methanopyri sp.

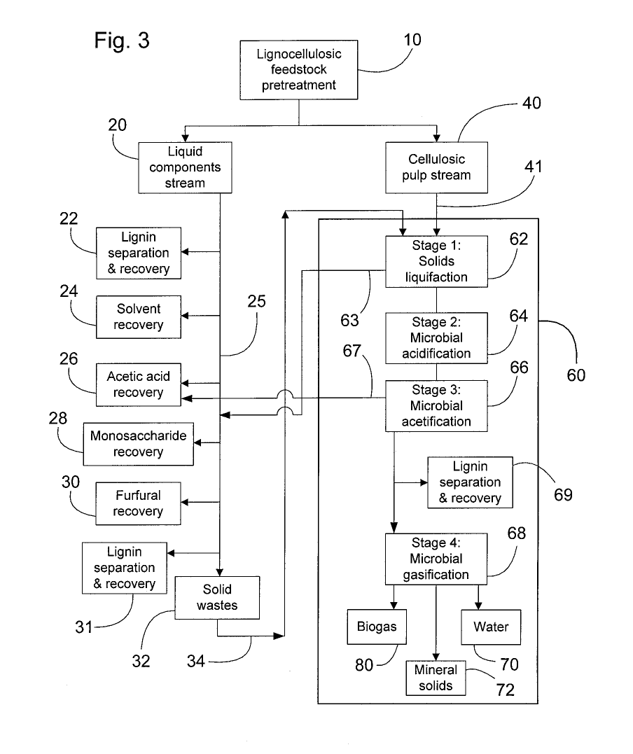

It is also optional to supply a portion of the liquefied stream of soluble monomers produced in the sludge tank 62 into the delignified stillage 25 in the liquid component processing stream (FIG. 3) for further processing and increased recovery of individual compounds from the lignocellulosic feedstock. Similarly, it is also optional to supply a portion of the acetic acid produced in the acetification vessel 66 to the acetic acid recovery component 26 of the liquid components processing stream. It is further optional to separate a novel class of lignins and lignin-derived polymers 69 from the liquid stream in the anaerobic digestion mode, or alternatively from any of the other three stages of the anaerobic digestion module.

The biogas produced from processed lignocellulosic feedstocks by the anaerobic digestion module of the present invention, can be fed directly into a power generation system as exemplified by a gas-fired combustion turbine. Combustion of biogas converts the energy stored in the bonds of the molecules of the methane contained in the biogas into mechanical energy as it spins a turbine. The mechanical energy produced by biogas combustion, for example, in an engine or micro-turbine may spin a turbine that produces a stream of electrons or electricity. In addition, waste heat from these engines can provide heating for the facility's infrastructure and/or for steam and/or for hot water for use as desired in the other modules of the present invention.

However, a problem with anaerobic digestion of semi-solid/solid waste materials is that the first step in the process, i.e., the hydrolysis of complex organic molecules comprising the semi-solid/solid waste materials into a liquid stream containing soluble monomers such as monosaccharides, amino acids and fatty acids, is typically lengthy and variable, while the subsequent steps, i.e., acidification, acetification, and biogas production proceed relatively quickly in comparison to the first step. Consequently, such lengthy and variable hydrolysis in the first step of anaerobic may result in insufficient amounts of biogas production relative to the facility's requirements for power production and/or steam and/or hot water. Accordingly, another embodiment of the present invention, as illustrated in FIGS. 1 and 2, controllably provides a portion of the monosccharide sugar stream produced during saccharification of cellulosic pulp 42 to the acidification tank 64 of the anaerobic digestion module 60 to supplement the supply of soluble monosaccharides hydrolyzed from semi-solid/solid materials delivered to the sludge tank 62. It is optional to also supply or alternatively to supply a portion of the monosccharide sugar stream 42 to the sludge tank 62.

Those skilled in these arts will understand that the processes and systems for configuring a 4-stage anaerobic digestion module as disclosed herein, for communicating and cooperating with lignocellulosic feedstock pre-treatment and processing systems e.g., cellulosic ethanol production, provides the operators of such lignocellulosic processing systems with new processes and systems that can be incorporated into their systems for one or more of: (a) improving the recovery of valuable extractives such as lignins, furfural and sugar streams from their feedstocks, (b) minimizing/eliminating the efflux of semi-solid/solid waste materials from their processes, (c) increasing the throughput rate of feedstock through their systems by manipulating the routing of sugar streams to and from the anaerobic digestion system of the present invention as disclosed herein, and (d) in the case where the interest may be primarily in optimizing the efficiency of a lignin biorefinery, the cellulosic pulp stream produced during the pre-treatment of the lignocellulosic feedstock may be delivered directly to the first-stage sludge tank of the anaerobic digestion system as disclosed herein.

1. A process for concurrent production of lignins, cellulosic material, fuel alcohol and biogas from a lignocellulosic feedstock, the process comprising the steps of:
pretreating the lignocellulosic feedstock to produce at least a solubilised liquid components stream comprising lignins and lignin-derived compounds, and an amorphous delignified solids output stream comprising cellulosic pulp;
separating the solubilised liquid components stream and the amorphous solids output stream;
further processing the solubilised liquid components stream to separate and recover therefrom at least lignins, lignin-derived compounds, and a semi-solid waste material;
further processing the amorphous solids output stream to hydrolyze the cellulosic pulp into a liquid stream comprising glucose, fermenting the liquid glucose stream to produce a beer, distilling the beer to recover therefrom a fuel-grade alcohol and a waste material comprising a stillage; and
anaerobically digesting the semi-solid waste material from the solubilised liquid components stream and the waste material from the amorphous solids output stream to produce a biogas therefrom, wherein the anaerobic digestion comprises the steps of:
first, liquefying the waste materials thereby producing a first liquid stream comprising monosaccharide sugars;
second, acidifying the first liquid stream thereby producing a second liquid stream comprising organic acids;
third, acetifying the second liquid stream thereby producing a third liquid stream comprising acetic acid; and
fourth, microbially converting the acetic acid to a biogas mixture comprising at least methane and carbon dioxide.

2. The process according to claim 1, wherein pretreating the lignocellulosic feedstock comprises physico-chemically digesting lignocellulosic feedstock with an aqueous organic solvent thereby extracting component parts therefrom into the solubilised liquid components stream.

3. The process according to claim 2, wherein the organic solvent comprises at least one solvent further defined as a short-chain alcohol, organic acid or ketones.

4. The process according to claim 3, wherein the organic solvent comprises at least one short-chain alcohol further defined as a methanol, ethanol, butanol, propanol, or aromatic alcohol.

5. The process according to claim 3, wherein the organic solvent comprises at least acetone.

6. The process according to claim 2, wherein the organic solvent is provided with a catalyst further defined as an inorganic acid or organic acid.

7. The process according to claim 1, wherein at least two classes of lignins are separated and recovered from the solubilised liquid components stream.

8. The process according to claim 1, wherein at least three classes of lignins are separated and recovered from the solubilised liquid components stream.

9. The process according to claim 1, wherein at least one class of lignins is separated and recovered from the amorphous solids output stream.

10. The process according to claim 1, wherein a portion of the liquid glucose stream hydrolyzed from the cellulosic pulp is controllably provided to at least one of the first step of anaerobic digestion and the second step of anaerobic digestion.

11. The process according to claim 1, where a portion of first liquid stream produced during anaerobic digestion is controllably provided to the solubilised liquid components stream during processing of said solubilised liquid components stream.

12. The process according to claim 1, where a portion of the second liquid stream produced during anaerobic digestion is controllably provided to the solubilised liquid components stream during processing of said solubilised liquid components stream.

13. The process according to claim 1, wherein the amorphous de-lignified solids output stream comprising cellulosic pulp is anaerobically digested.

14. The process according to claim 1, wherein the first step of anaerobic digestion is provided with a microbial inoculum comprising at least one strain of Enterobacter sp.

15. The process according to claim 1, wherein the second step of anaerobic digestion is provided with a microbial inoculum comprising at least one strain of Bacillus sp., Lactobacillus sp. or Streptococcus sp.

16. The process according to claim 1, wherein the third step of anaerobic digestion is provided with a microbial inoculum comprising at least one strain of Acetobacter sp., Gluconobacter sp., or Clostridium sp.

17. The process according to claim 1, wherein the fourth step of anaerobic digestion is provided with a microbial inoculum comprising at least one strain of Methanobacteria sp., Methanococci sp., or Methanopyri sp.

18. The process according to claim 1, wherein said process is a batch process.

19. The process according to claim 1, wherein said process is a continuous throughput process.

20. A process for concurrent production of lignins and biogas from a lignocellulosic feedstock, the process comprising the steps of:
pretreating the lignocellulosic feedstock to produce at least a solubilised liquid components stream comprising lignins and lignin-derived compounds, and an amorphous delignified solids output stream comprising cellulosic pulp;
separating the solubilised liquid components stream and the amorphous solids output stream;
further processing the solubilised liquid components stream to separate and recover therefrom at least lignins, lignin-derived compounds, and a semi-solid waste material; and
anaerobically digesting the semi-solid waste material from the solubilized liquid components stream and the amorphous solids output stream to produce a biogas therefrom, wherein the anaerobic digestion comprises the steps of:
first, liquefying the waste materials thereby producing a first liquid stream comprising monosaccharide sugars;
second, acidifying the first liquid stream thereby producing a second liquid stream comprising organic acids;
third, acetifying the second liquid stream thereby producing a third liquid stream comprising acetic acid; and
fourth, microbially converting the acetic acid to a biogas mixture comprising at least methane and carbon dioxide.

21. A system for concurrent production of lignins, fuel alcohol, and biogas from a lignocellulosic feedstock, the system comprising:
equipment configured for controllably receiving, commingling and processing therein a lignocellulosic feedstock and an organic solvent, and further configured to controllably provide at least a first output stream comprising amorphous de-lignified solids and a second output stream comprising a spent organic solvent comprising solubilized and suspended organic matter, said spent organic solvent containing therein lignins and lignin-derived compounds;
equipment configured for controllably receiving and hydrolyzing therein said amorphous solids stream, and for controllably discharging a stream of hydrolysate therefrom;
equipment configured for controllably separating said hydrolysate stream into at least a first hydrolysate stream and a second hydrolysate stream;
equipment configured for controllably delivering said first hydrolysate stream into a fuel alcohol production system; and
equipment configured for controllably delivering said second hydrolysate stream into an anaerobic digestion system.

22. The system according to claim 21, further defined as configured to controllably deliver said first output stream comprising amorphous de-lignified solids to an anaerobic digestion system.

23. The system according to claim 21, further comprising:
equipment configured for controllably receiving and de-lignifying therein said spent organic solvent stream, for controllably separating lignins from said de-lignified spent solvent stream, and for controllably discharging therefrom said de-lignified spent solvent stream; and
equipment configured for controllably separating the de-lignified spent solvent stream into a first de-lignified spent solvent stream manipulably deliverable into an anaerobic digestion system, and a second de-lignified spent solvent stream.

24. The system according to claim 21, further comprising equipment configured for controllably separating and discharging selected organic compounds therefrom into said second de-lignified spent solvent stream.

25. The system according to claim 21, further comprising equipment configured for controllably delivering said second de-lignified spent solvent stream into a fuel alcohol production system.

26. The system according to claim 21, wherein said process is a batch process.

27. The system according to claim 21, wherein said process is a continuous throughput process.
